(19) United States
(12) Patent Application Publication
(10) Pub. No.: US 2014/0296820 A1
(43) Pub. Date: Oct. 2, 2014

(54) CLOTH DIAPER

(71) Applicant: Christina L. Malone, Grand Rapids, MI (US)

(72) Inventor: Christina L. Malone, Grand Rapids, MI (US)

(21) Appl. No.: 14/242,414

(22) Filed: Apr. 1, 2014

Related U.S. Application Data
(60) Provisional application No. 61/807,458, filed on Apr. 2, 2013.

(51) Int. Cl. A61F 13/56

(52) U.S. Cl. CPC A61F 13/5644; A61F 2013/5672; A61F 2013/5677
USPC 604/385.201

(57) ABSTRACT

A cloth diaper includes a size adjustable impermeable outer layer, and an elongated absorbent insert permanently attached only at one end to the inside of said outer layer, near the upper edge of the outer layer. The absorbent insert extends a substantial distance beyond the opposite upper edge of said outer layer. Preferably, there is an absorbent layer attached to the outer layer, between the outer layer and the insert.

### CROSS REFERENCE TO RELATED APPLICATION

This application claims priority to U.S. Provisional Application Ser. No. 61/807,458, filed Apr. 2, 2013, entitled CLOTH DIAPER.

### BACKGROUND

The present invention relates to cloth diapers. Cloth diaper manufacturers have designed diapers that have an absorbent insert that is attached to both the reside front and back of the diaper, wherein excess fabric may be folded to accommodate male or female. Other cloth diapers have an absorbent insert that is attached with some form of fastener at either the front or back of the diaper, which allows the user to add additional absorbency to the diaper. In some diapers, the snap in and separable insert is the only absorbent member in the diaper. The additional inserts may be removed during laundering to aid in the washing and drying. Another variation is a cloth diaper with so absorbent insert which is sewn into the inside of the diaper on three sides, forming a pocket, and including an extending tail which is folded into the pocket for use.

Size adjustable diapers are available, in which the length of the diaper from the front edge to the back edge can be adjusted, by providing two or three rows of like fasteners spaced from at least one row of mating fasteners. By folding a portion of the length of the diaper on itself, and snapping mating fasteners together to hold the fold, the size of the diaper is adjusted.

### SUMMARY OF THE INVENTION

In the present invention, a cloth diaper includes a size adjustable impermeable outer layer, and an elongated absorbent insert permanently attached only at one end to the inside of said outer layer, near the upper edge of the outer layer. The absorbent insert extends a substantial distance beyond the opposite upper edge of said outer layer. Preferably, there is an absorbent layer attached to the outer layer, between the outer layer and the insert.

### BRIEF DESCRIPTION OF THE DRAWINGS

FIG. 1 is a front view of a preferred embodiment adjustable diaper at its largest size;

FIG. 2 is a plan view of the interior side of the diaper of FIG. 1 opened flat, arranged with its back portion at the top of figure, its front portion at the bottom of the figure, and the absorbent insert fully extending;

FIG. 3 is a plan view of the interior side of the diaper, showing the absorbent insert folded over once;

FIG. 4 is a plan view of the interior side of the diaper, showing the absorbent insert folded over twice at middle of the diaper, which will be the bottom of the diaper as worn;

FIG. 5 is a plan view of the interior side of the diaper, showing the absorbent insert folded over twice toward the front of the diaper;

FIG. 6 is a front view of the diaper adjusted to a smaller size.

FIG. 7 is plan view of the interior of the adjusted smaller diaper of FIG. 6 opened flat, with the absorbent insert folded over twice to accommodate the smaller size of the diaper; and

FIG. 8 is a front view of the adjusted smaller diaper as it will look when worn.

### DESCRIPTION OF THE PREFERRED EMBODIMENTS

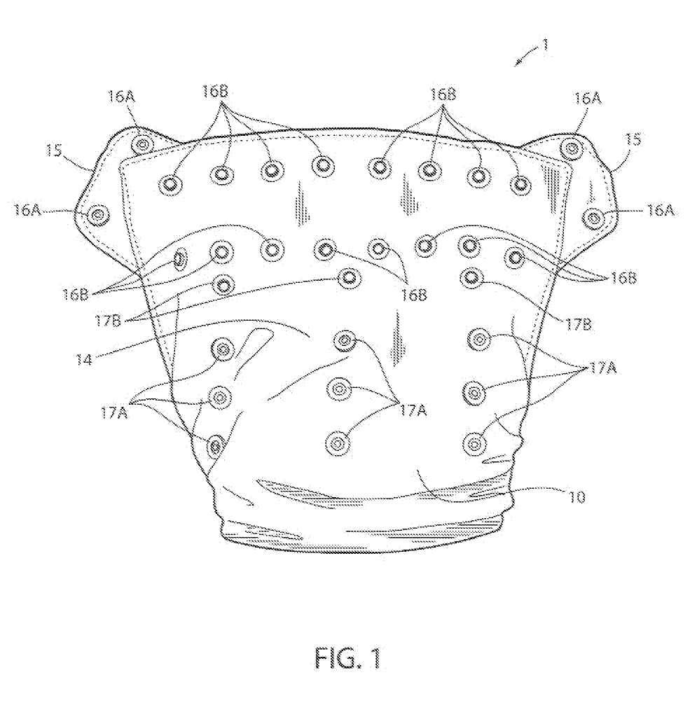

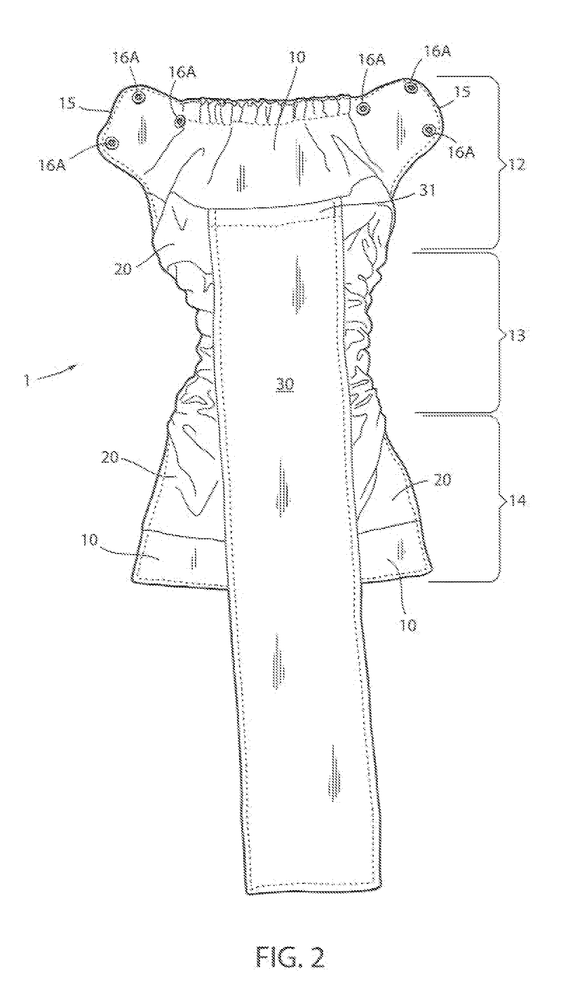

The preferred embodiment diaper 1 shown in the drawings has a water proof outer layer 10, to which is attached to absorbent layer 20, which comprises of one or more layers of an absorbent material (FIGS. 1 and 2). The term “layer” as used herein includes an integrated layer which may itself be made up of two or several layers of fabric. Inner absorbent layer 20 is dimensioned to encompass the area of the “wet zone” of the diaper. As is well known to every parent, the wet zone ordinarily extends from near the front or back upper edge of the diaper to near the opposite upper edge. At one end of interior of the diaper, specifically at an end of the wet zone, an absorbent insert 30 is permanently attached to the water proof outer layer. As shown, insert 30 is attached to at the top of the wet zone, which will be the top of the rear of the wet zone when the diaper is worn.

Absorbent insert 30 exceeds the length of the diaper by a substantial distance, and may be folded to meet baby's needs. The term substantial distance means the absorbent insert when folded over once when when the diaper is adjusted to its largest size, provides two layers of added absorbency over the wet zone of the diaper. Preferably, absorbent insert 30 is about twice the length of the wet zone of the diaper, when the diaper is adjusted to its largest size, and most preferably at least twice the length of the wet zone. As can be seen from the drawings, absorbent insert is made of material such that either side of absorbent insert 30 can be placed against the baby's skin.

Outer layer 10 is made of a water impervious material. It is sewn to absorbent layer 20, which is comprised of one or more layers 20 of absorbent material. The waterproof layer 10 extends down the back, bottom and front sides 12, 13 and 14 respectively, of the diaper to prevent wicking of moisture out of absorbent layer 20, and absorbent insert 30. Elastic is encased around the top edge 11 of the back or rear portion 12 of the diaper (FIG. 2) to help prevent “blow-outs”. Elastic is sewn to the inner seam around both curvilinear edges of the center or bottom portions 13 of the diaper 1.

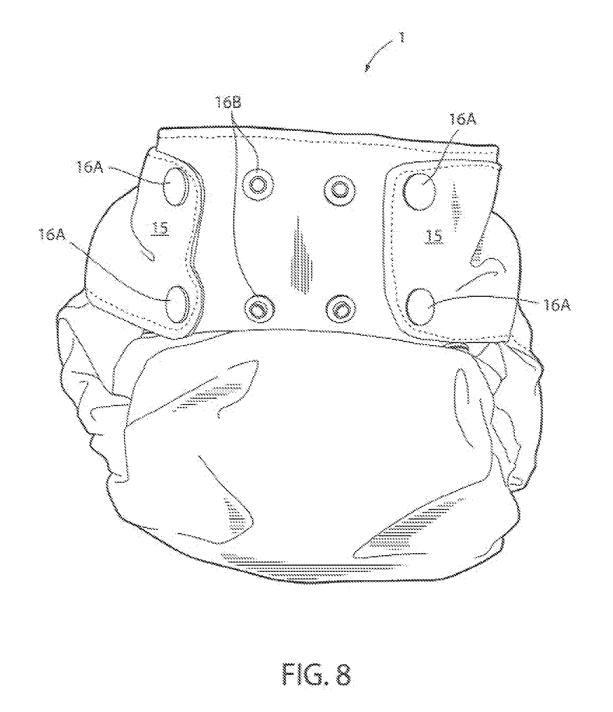

There are waterproof tabs 15 that extend out from the upper back portion 12 of the diaper and wrap around the front 14 of the diaper, using fasters 16A to securely fasten to mating fasteners 16B at the front 14 of the diaper, thereby securing the diaper around the waist of the wearer. In the preferred embodiments, fasteners 16A and 16B are mating male and female snaps. There are two horizontal rows of eight mating snaps 16B each, on the front 14 of outer cover 10. The two rows of snaps 16B are spaced to correspond to the spacing of snaps 16A on each of tabs 15. As shown, snaps 16A are female snaps, and snaps 16B are male snaps. Snaps multiple snaps 16B served as waist adjustment snaps, in that the diaper tabs 15 can be fastened to different pairs of snaps 16B, in order to accommodate varying baby waist sizes. In this way, diaper 10 can be used for babies of many different sizes, and indeed can be dimensioned to fit substantially all sizes. By comparing FIG. 8 to FIG. 1, one can see that in FIG. 8, the female snaps 16A on tabs 15 have been secured to the third pair of male snaps 16B in from each edge, leaving only two pair of the male snaps 16B visible in FIG. 8.

Inner absorbent layer or layers 20 are preferably sewn to outer layer 10. Absorbent insert 30 is preferably comprised of multiple layers of absorbent fabric and is attached only along an end edge 31 to the back portion 12 of diaper 1, where the outer waterproof layer and the inner absorbent fabric layer 20 meet.

The rise of the diaper is adjusted by using mating rise adjustment fasteners 17A and 17B that are reinforced and located across the front 14 of outer waterproof layer 10 (FIG. 1). In the preferred embodiment, fasteners 17A and 17B are snaps. There is one row of three male snaps 17B toward the upper portion of front 14, just below the bottom row of eight male snaps which are available for mating with the bottom female snap 17A on tabs 15. There are three rows of three each female snaps 17A, each spaced progressively further below the upper row of three male snaps 17B. By folding front 14 upon itself, and fastening one of the three rows of female snaps 17A to the upper row of three male snaps 17B, diaper 1 can be adjusted to three different sizes, in terms of the “rise.” By comparing FIG. 1 to FIG. 6, one can see that the bottom row of female snaps 17A has been secured to the row of three male snaps 16B, thereby shortening the rise of diaper 1 to its shortest position. One can adjust the rise to two intermediate positions by using either of the other two rows of three female snaps 17A.

The long “tongue-like” permanently attached absorbent insert 30 allows for all the absorbency that is necessary, while providing optimal exposure to water and air during the laundering process. Since only one end of the insert is connected to the interior of the diaper, there is less bulk on smaller babies and the preferred amount of absorbency where necessary for male and/or female.

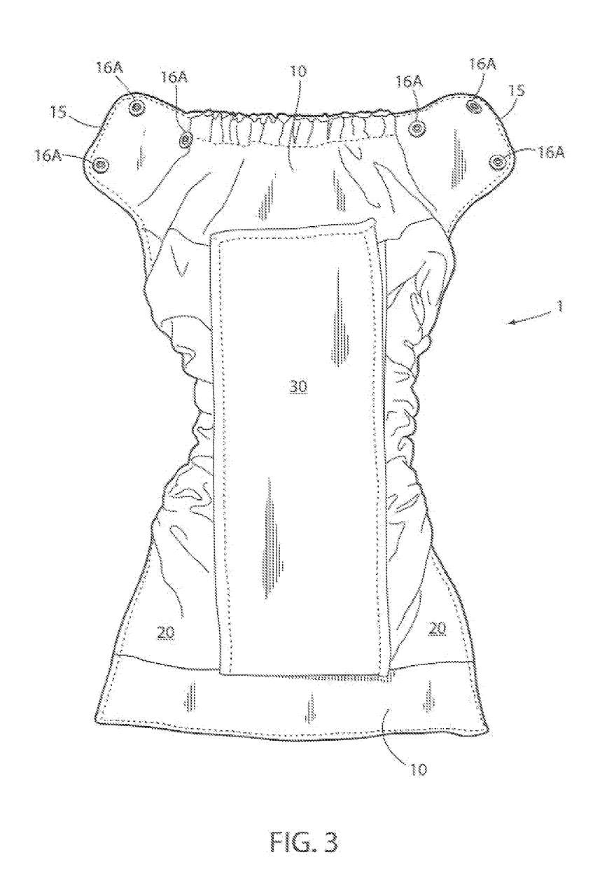

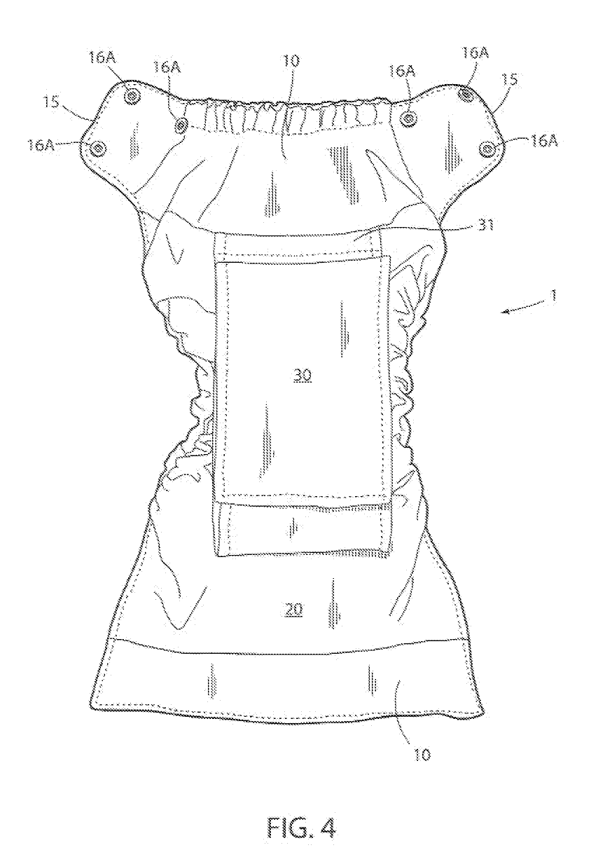

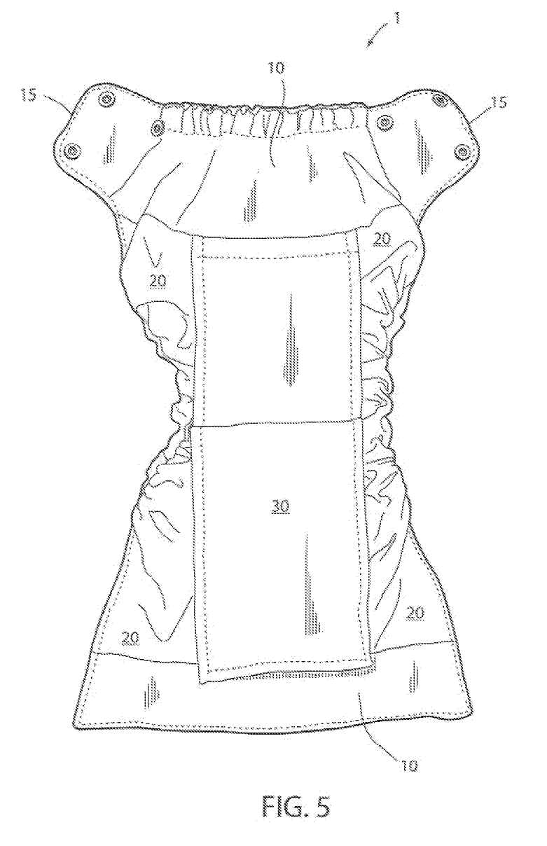

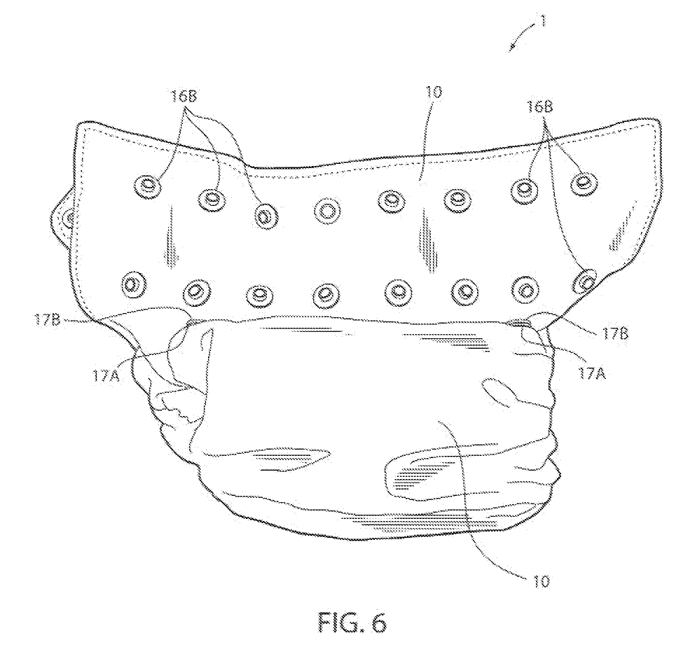

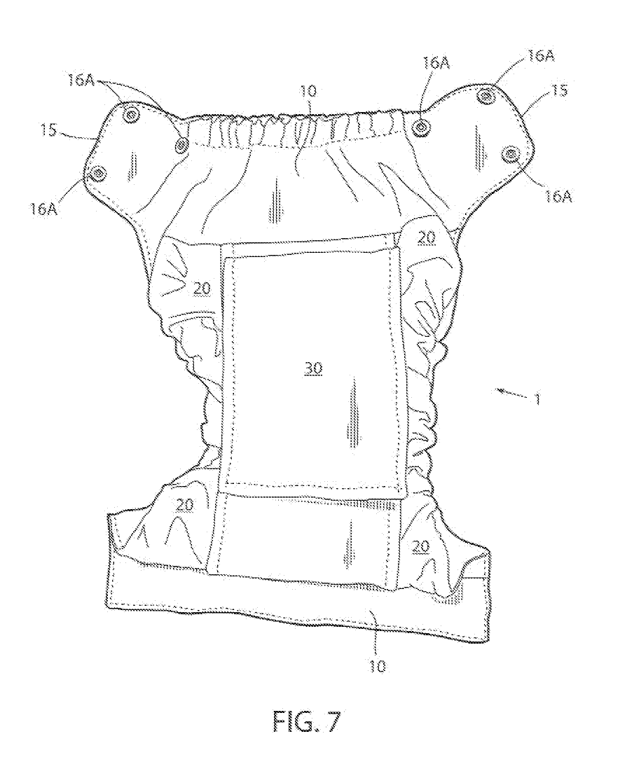

In use, diaper 1 should be laid out flat and the attached insert 30 folded with more fabric in the middle for females (FIG. 4), more in the front for males (FIG. 5), or distributed evenly inside the diaper (FIG. 3). The front rise snaps 17A and 17B should be fastened so that the front of the diaper is close in proximity to the wearer's navel. When set to one of the smaller rise settings (FIG. 6), it will be advantageous for the insert to be folded with more fabric towards the middle or back of the diaper to reduce bulkiness (FIG. 7). Once the insert is positioned and the rise appropriately set, one lays the baby on the diaper and fastens tabs 15 to the front snaps 16B so as to hold the diaper securely around the baby's waist.

The present diaper provides a one-size, all-in-one diaper that washes cleaner and dries faster than other one-size, all-in-one diapers on the market. Most one-size, all-in-one diapers use too many layers of fabric, in too close of proximity with each other. Such diapers take multiple drier cycles to completely dry, and generally result in stinky, damp diapers. The present diaper is easy to use, and allows the absorbency to be adjusted to accommodate the needs of both male and female.

Finally, the present diaper is trimmer than other one-size, all-in-one diapers without eliminating of absorbency. Even in its largest size, permanently attached insert 30 provides two additional layers of absorbency, in addition to absorbent layer 20. In FIGS. 4 and 5, three additional layers of absorbency are provided in critical locations. In the diaper as adjusted to its smallest size, three additional layers of absorbency are provided. Thus for most applications one has at least four layers of absorbency, at least in the critical locations. Yet in the washing and drying cycle, there is realty only one layer of absorbency being washed and dried.

Of course, it is understood that the foregoing describes preferred embodiments of the invention, and that changes and alterations can be made without departing from the spirit and scope of the invention as set forth in the appended claims.

1. A size adjustable cloth diaper comprising: a size adjustable water impermeable outer layer, providing a front, bottom and back to said diaper; an elongated absorbent insert permanently attached only at one end to the inside of said outer layer, near an upper edge of said front or back of said outer layer, said absorbent insert extending a substantial distance beyond the opposite upper edge of said outer layer.

2. The size adjustable cloth diaper of claim 1 in which there is an absorbent layer attached to said outer layer, between said outer layer and said insert.

3. The size adjustable cloth diaper of claim 2 in which said absorbent insert is about twice the length of the wet zone of the diaper, when the diaper is adjusted to its largest size.

4. The size adjustable cloth diaper of claim 3 in which said absorbent insert is at least twice the length of said wet zone.

5. The size adjustable cloth diaper of claim 4 in which said absorbent insert is made of a material which allows either side of said absorbent insert to be placed against the baby's skin.

6. The size adjustable cloth diaper of claim 3 in which the rise of said diaper is adjusted by providing mating rise adjustment fasteners at multiple levels across the front of said outer water impermeable layer, such that said fasteners can be secured at different levels to facilitate adjustment of the rise of said diaper.

7. The size cloth diaper of claim 6 in which there at least one upper rise adjustment fastener is located uppermost on the front of said outer water impermeable layer, and fasteners which mate with said upper fastener are located at multiple levels below said at least one upper fastener.

8. The size adjustable cloth diaper of claim 7 in which there are three successively lower levels of said rise adjustment fasteners which mate with said at least one upper rise adjustment fastener.

9. The size adjustable cloth diaper of claim 8 in which said rise adjustment fasteners comprise mating snaps.

10. The size adjustable cloth of claim 6 comprising: said outer water impermeable layer including securing tabs at each upper rear corner of said diaper as worn; each said tab including at least one tab fastener; a plurality of waist adjusting fasteners being located at multiple locations horizontally across the front of said outer water impermeable layer, each being matable with said tab fasteners, whereby said tabs can be secured at any of multiple locations across said front of said outer water impermeable layer.

11. The size adjustable cloth diaper of claim 10 in which said tab fasteners and said waist adjusting fasteners comprise mating snaps.

12. The size adjustable cloth diaper of claim 10 in which said absorbent insert is secured near the upper edge of said back of said diaper.

13. A size adjustable cloth diaper comprising: a size adjustable water impermeable outer layer, providing a front, bottom and back to said diaper; an absorbent layer attached to said outer layer, extending from near the upper edge of said front of said outer layer to near the upper edge of said back of said outer layer, encompassing the wet zone of said diaper; and elongated absorbent insert permanently attached only at one end to the inside of said outer layer and said absorbent layer, at an upper edge of said absorbent layer at either said front or said back of said outer layer, said absorbent insert extending a substantial distance beyond the opposite upper edge of said outer layer; said absorbent insert being about twice the length of the wet zone of said diaper, when said diaper is adjusted to its largest size; mating rise adjustment fasteners being located at multiple levels across the front of said outer water impermeable layer, such that said rise adjustment fasteners can be secured at different levels to facilitate adjustment of the rise of said diaper; said outer water impermeable layer including securing tabs at each upper rear corner of said diaper as worn; each said tab including at least one tab fastener; a plurality of waist adjusting fasteners being located at multiple locations horizontally across the front of said outer water impermeable layer, each being matable with said tab fasteners, whereby said tabs can be secured at any of multiple locations across said front of said outer water impermeable layer.

14. The size adjustable cloth diaper of claim 13 in which said absorbent insert is an least twice the length of said wet zone.

15. The size adjustable cloth diaper of claim 14 in which said absorbent insert is made of a material which allows either side of said absorbent insert to be placed against the baby's skin.

16. The size adjustable cloth diaper of claim 15 in which there at least one upper rise adjustment fastener is located uppermost on the front of said outer water impermeable layer, and fasteners which mate with said upper fastener are located at multiple levels below said at least one upper fastener.

17. The size adjustable cloth diaper of claim 16 in which there are three successively lower levels of said rise adjustment fasteners which mate with said at least one upper rise adjustment fastener.

18. The size adjustable cloth diaper of claim 17 in which said rise adjustment fasteners comprise mating snaps.

19. The size adjustable cloth diaper of claim 13 in which said tab fasteners and said waist adjusting fasteners comprise mating snaps.

20. The size adjustable cloth diaper of claim 13 in which said absorbent insert is secured near the upper edge of said back of said diaper.
